(19) United States
(12) Patent Application Publication
(10) Pub. No.: US 2005/0281887 A1
(43) Pub. Date: Dec. 22, 2005

(54) Fluid conditioning system

(76) Inventors: Ioana M. Rizoiu, San Clemenle, CA (US); Jeffrey W. Jones, Robertson, WY (US)

Correspondence Address: STOUT, UXA, BUYAN & MULLINS LLP, 4 VENTURE, SUITE 300, IRVINE, CA, 92618, US

(21) Appl. No.: 11/033,044

(22) Filed: Jan. 10, 2005

Related U.S. Application Data
(63) Continuation-in-part of application No. 10/435,325, filed on May 9, 2003.
Application No. 10/435,325 is a division of application No. 09/997,550, filed on Nov. 27, 2001, now Pat. No. 6,561,803.
Application No. 09/997,550 is a continuation of application No. 09/256,697, filed on Feb. 24, 1999, now Pat. No. 6,350,123.
Application No. 09/256,697 is a continuation-in-part of application No. 08/985,513, filed on Dec. 5, 1997, now abandoned.
Application No. 08/985,513 is a continuation of application No. 08/522,503, filed on Aug. 31, 1995, now Pat. No. 5,741,247.
(60) Provisional application No. 60/535,110, filed on Jan. 8, 2004.

(51) Int. Cl. A61K 33/00; A61K 7/18; A61H 21/00

(52) U.S. Cl. 424/600; 607/86

(57) ABSTRACT

A fluid conditioning system is adapted to condition the fluid used in medical and dental cutting, irrigating, evacuating, cleaning, and drilling operations. The fluid may be conditioned by adding flavors, antiseptics and/or tooth whitening agents such as peroxide, medications, and pigments. In addition to the direct benefits obtained from introduction of these agents, the laser cutting properties may be varied from the selective introduction of the various agents.

### CROSS-REFERENCE TO RELATED APPLICATIONS

This application claims the benefit of U.S. Provisional Application No. 60/535,110, filed Jan. 8, 2004 and entitled FLUID CONDITIONING SYSTEM, the contents of which are expressly incorporated herein by reference. This application is also a continuation-in-part of U.S. application Ser. No. 10/435,325, filed May 9, 2003, which is a divisional of U.S. application Ser. No. 09/997,550, filed Nov. 27, 2001, issued as U.S. Pat. No. 6,561,803, which is a continuation of U.S. application Ser. No. 09/256,697, filed Feb. 24, 1999, issued as U.S. Pat. No. 6,350,123, which is a continuation-in-part of U.S. application Ser. No. 08/985,513, filed Dec. 5, 1997, now abandoned, which is a continuation of U.S. application Ser. No. 08/522,503, filed Aug. 31, 1995, issued as U.S. Pat. No. 5,741,247, the contents of all which are expressly incorporated herein by reference.

### BACKGROUND OF THE INVENTION

1. Field of the Invention

The present invention relates generally to medical cutting, irrigating, evacuating, cleaning, and drilling techniques and, more particularly to a device for cutting both hard and soft materials and a system for introducing conditioned fluids into the cutting, irrigating, evacuating, cleaning, and drilling techniques.

2. Description of Related Art

A prior art dental/medical work station 11 is shown in FIG. 1. A vacuum line 12 and an air supply line 13 supply negative and positive pressures, respectively. A water supply line 14 and an electrical outlet 15 supply water and power, respectively. The vacuum line 12, the air supply line 13, the water supply line 14, and the power source 15 are all connected to the dental/medical (e.g., dental or medical) unit 16.

The dental/medical unit 16 may comprise a dental seat or an operating table, a sink, an overhead light, and other conventional equipment used in dental and medical procedures. The dental/medical unit 16 may provide, for example, water, air, vacuum and/or power to the instruments 17. These instruments may include, for example, an electrocauterizer, an electromagnetic energy source, a mechanical drill, a mechanical saw, a canal finder, a syringe, and/or an evacuator. Various other types, combinations, and configurations of dental/medical units 16 and subcomponents implementing, for example, an electromagnetic energy device operating with a spray, have also existed in the prior art, many or most of which may have equal applicability to the present invention.

The electromagnetic energy source is typically a laser coupled with a delivery system. The laser 18a and delivery system 19a, both shown in phantom, as well as any of the above-mentioned instruments, may be connected directly to the dental/medical unit 16. Alternatively, the laser 18b and delivery system 19b, both shown in phantom, may be connected directly to the water supply 14, the air supply 13, and the electric outlet 15. Other instruments 17 may be connected directly to any of the vacuum line 12, the air supply line 13, the water supply line 14, and/or the electrical outlet 15.

The laser 18 and delivery system 19 may typically comprise an electromagnetic cutter for dental use, although a variety of other types of electromagnetic energy devices operating with fluids (e.g., sprays) may also be used. An example of one of many varying types of conventional prior art electromagnetic cutters is shown in FIG. 2. According to this example of a prior art apparatus, a fiber guide tube 30, a water line 31, an air line 32, and an air knife line 33 (which supplies pressurized air) may be fed from the dental/medical unit 16 into the hand-held apparatus 34. A cap 35 fits onto the hand-held apparatus 34 and is secured via threads 36. The fiber guide tube 30 abuts within a cylindrical metal piece 37. Another cylindrical metal piece 38 is a part of the cap 35. When the cap 35 is threaded onto the hand-held device 34, the two cylindrical metal tubes 37 and 38 are moved into very close proximity of one another. The pressurized air from the air knife line 33 surrounds and cools the laser as the laser bridges the gap between the two metal cylindrical objects 37 and 38. Air from the air knife line 33 flows out of the two exhausts 39 and 41 after cooling the interface between elements 37 and 38.

The laser energy exits from the fiber guide tube 42 and is applied to a target surface within the patient's mouth, according to a predetermined surgical plan. Water from the water line 31 and pressurized air from the air line 32 are forced into the mixing chamber 43. The air and water mixture is very turbulent in the mixing chamber 43, and exits this chamber through a mesh screen with small holes 44. The air and water mixture travels along the outside of the fiber guide tube 42, and then leaves the tube 42 and contacts the area of surgery. The air and water spray coming from the tip of the fiber guide tube 42 helps to cool the target surface being cut and to remove materials cut by the laser.

Water is generally used in a variety of laser cutting operations in order to cool the target surface. Additionally, water is used in mechanical drilling operations for cooling the target surface and removing cut or drilled materials therefrom. Many prior art cutting or drilling systems use a combination of air and water, commonly combined to form a light mist, for cooling a target surface and/or removing cut materials from the target surface.

The use of water in these and other prior art systems has been somewhat successful for purposes of, for example, cooling a target surface or removing debris therefrom. These prior art uses of water in cutting and drilling operations, however, may not have allowed for versatility, outside of, for example, the two functions of cooling and removing debris. In particular, during cutting or drilling operations, including those using systems with water, for example, for cooling or removing debris from a target surface, medication treatments, preventative measure applications, and aesthetically pleasing substances, such as flavors or aromas, may have not been possible or used. A conventional drilling operation may benefit from the use of an anesthetic near the drilling operation, for example, but during this drilling operation only water and/or air are often used. In the case of a laser cutting operation, a disinfectant, such as iodine, could be applied to the target surface during drilling to guard against infection, but this additional disinfectant may not be commonly applied during such laser cutting operations. In the case of an oral drilling or cutting operation, unpleasant tastes or odors may be generated, which may be unpleasing to the patient. The common use of only water during this oral procedure does not mask the undesirable taste or odor. A need has thus existed in the prior art for versatility of applications and of treatments during drilling and cutting procedures.

Compressed gases, pressurized air, and electrical motors are commonly used to provide the driving force for mechanical cutting instruments, such as drills, in dentistry and medicine. The compressed gases and pressurized water are subsequently ejected into the atmosphere in close proximity to or inside of the patient's mouth and/or nose. The same holds true for electrically driven turbines when a cooling spray (air and water) is typically ejected into the patient's mouth, as well. These ejected fluids commonly contain vaporous elements of tissue fragments, burnt flesh, and ablated or drilled tissue. This odor can be quite uncomfortable for the patient, and can increase trauma experienced by the patient during the drilling or cutting procedure. In a such a drilling or cutting procedure, a mechanism for masking the smell and the odor generated from the cutting or drilling may be advantageous.

Another problem exists in the prior art with bacteria growth on surfaces within a dental operating room. The interior surfaces of air, vacuum, and water lines of the dental/medical unit, for example, are subject to bacteria growth. In waterlines the bacterial growth is part of the biofilm forming on the inside of the waterline tubing. Additionally, the air and water used to cool the tissue being cut or drilled within the patient's mouth is often vaporized into the air to some degree. This vaporized air and water condensates on surfaces of the dental equipment within the dental operating room. These moist surfaces can also promote bacteria growth, which is undesirable. A system for reducing the bacteria growth within air, vacuum, and water lines, and for reducing the bacteria growth resulting from condensation on exterior surfaces, is needed to reduce sources of contamination within a dental operating room.

### SUMMARY OF THE INVENTION

The fluid conditioning system of the present invention is adaptable to most existing medical and dental cutting, irrigating, evacuating, cleaning, and drilling apparatuses. Flavored fluid is used in place of regular tap water or other types of water such as distilled, deionized, sterile, or water with a controlled number of colony forming units (CFU) per milliliter, etc., during drilling operations. In the case of a laser surgical operation, electromagnetic energy is focused in a direction of the tissue to be cut, and a fluid router routes flavored fluid in the same direction. The flavored fluid may appeal to the taste buds of the patient undergoing the surgical procedure, and may include any of a variety of flavors, such as a fruit flavor or a mint flavor. In the case of a mist or air spray, scented air may be used to mask the smell of burnt or drilled tissue. The scent may function as an air freshener, even for operations outside of dental applications.

The fluids used for cooling a surgical site and/or removing tissue may further include an ionized solution, such as a biocompatible saline solution, and may further include fluids having predetermined densities, specific gravities, pH levels, viscosities, or temperatures, relative to conventional tap water. Additionally, the fluids may include a medication, such as an antibiotic, a steroid, an anesthetic, an anti-inflammatory, an antiseptic or disinfectant, adrenaline, epinephrine, or an astringent. The fluid may also include vitamins, herbs, or minerals. Still further, the fluid may include a tooth-whitening agent that is adapted to whiten a tooth of a patient. The tooth-whitening agent may comprise, for example, a peroxide, such as hydrogen peroxide, urea peroxide, or carbamide peroxide. The tooth-whitening agent may have a viscosity on an order of about 1 to 15 centipoises (cps).

Introduction of any of the above-mentioned conditioning agents to the conventional water (or other types of water such as distilled, deionized, sterile, or water with a controlled number of CFU/ml, etc.) of a cutting or drilling operation may be controlled by a user input. Thus, for example, a user may adjust a knob or apply pressure to a foot pedal in order to introduce iodine into the water after a cutting operation has been performed. The amount of conditioning applied to the air, water, or mist may be a function of the position of the foot pedal, for example.

According to one broad aspect of the present invention, a mist of atomized particles is placed into a volume of air above the tissue to be cut, and a source of electromagnetic energy, such as a laser, is focused into the volume of air. The electromagnetic energy has a wavelength, which is substantially absorbed by the atomized particles in the volume of air. Disruptive (e.g., mechanical) cutting forces can be imparted onto the tissue. In certain implementations, absorption of the electromagnetic energy by the atomized particles causes the atomized particles to explode and impart disruptive cutting forces onto the tissue. According to this effect, the electromagnetic energy source does not directly cut the tissue but, rather, the exploded fluid particles are used to cut the tissue. In other embodiments, exploding fluid particles may not affect at all, or may affect a percentage but not all of, the cutting of tissue. Examples of such embodiments are disclosed in U.S. application Ser. No. ______, filed Jan. 10, 2005 and entitled ELECTROMAGNETIC ENERGY DISTRIBUTIONS FOR ELECTROMAGNETICALLY INDUCED DISRUPTIVE CUTTING, the entire contents of which are incorporated herein by reference to the extent compatible and not mutually exclusive. These fluid particles may be conditioned with flavors, scents, ionization, medications, disinfectants, and other agents, as previously mentioned.

Since the electromagnetic energy is focused directly on the atomized, conditioned fluid particles, the cutting forces are changed, depending upon the conditioning of the atomized fluid particles. The disruptive cutting efficiency can be proportional (related) to the absorption of the electromagnetic energy by the fluid spray. The absorption characteristic can be modified by changing the fluid composition. For example, introduction of a salt into the water before atomization, resulting in an ionized solution, will exhibit slower cutting properties than does regular water. This slower cutting may be desirable, or the laser power may be increased to compensate for the ionized, atomized fluid particles. Additionally, the atomized fluid particles may be pigmented to either enhance or retard absorption of the electromagnetic energy, to thereby additionally control the cutting power of the system. Two sources of fluid may be used, with one of the sources having a pigment and the other not having a pigment.

Another feature of the present invention places a disinfectant in the air, mist, or water used for dental or surgical applications. This disinfectant can be periodically routed through the air, mist, or water lines to disinfect the interior surfaces of these lines. This routing of disinfectant can be performed between patients, daily, or at any other predetermined intervals. A mouthwash may be used, for example, during or at the end of procedures to both clean the patient's-mouth and clean the air and water tubes.

According to another feature of the present invention, when disinfectant is routed through the lines during a medical procedure, the disinfectant stays with the water or mist, as the water or mist becomes airborne and settles on surrounding surfaces within the dental operating room. Bacteria growth within the lines, and from the condensation, is significantly attenuated, since the disinfectant kills, stops and/or retards bacteria growth inside the fluid (e.g., water) lines and/or on any moist surfaces.

The present invention, together with additional features and advantages thereof, may best be understood by reference to the following description taken in connection with the accompanying illustrative drawings.

Any feature or combination of features described herein are included within the scope of the present invention provided that the features included in any such combination are not mutually inconsistent as will be apparent from the context, this specification, and the knowledge of one of ordinary skill in the art.

Additional advantages and aspects of the present invention are apparent in the following detailed description and claims.

### BRIEF DESCRIPTION OF THE DRAWINGS

FIG. 1 illustrates a conventional dental/medical work station;

FIG. 2 is an example of one of many types of conventional optical cutter apparatuses;

FIG. 3 illustrates a dental/medical work station according to an embodiment of the present invention;

FIG. 4 is a schematic block diagram illustrating an electromagnetic cutter using conditioned fluid, according to one embodiment of the present invention;

FIG. 5a illustrates one embodiment of an electromagnetic cutter of the present invention;

FIG. 5b illustrates another embodiment of an electromagnetic cutter of the present invention;

FIG. 6a illustrates a mechanical drilling apparatus according to an implementation of the present invention;

FIG. 6b illustrates a syringe according to an implementation of the present invention;

FIG. 7 illustrates a fluid conditioning system according to an embodiment of the present invention;

FIG. 8 illustrates one embodiment of the fluid conditioning unit of the present invention;

FIG. 9 illustrates an air conditioning unit according to an embodiment of the present invention;

FIG. 10 is a schematic block diagram illustrating an electromagnetically induced disruptive cutter according to an embodiment of the present invention;

FIG. 11 is an optical cutter with a focusing optic in accordance with an embodiment of the present invention;

FIG. 12 illustrates a control panel for programming a combination of atomized fluid particles according to an illustrated embodiment;

FIG. 13 is a plot of particle size versus fluid pressure in accordance with one implementation of the present invention;

FIG. 14 is a plot of particle velocity versus fluid pressure in accordance with one implementation of the present invention;

FIG. 15 is a schematic diagram illustrating a fluid particle, a source of electromagnetic energy, and a target surface according to an embodiment of the present invention;

FIG. 16 is a schematic diagram illustrating a “grenade” effect according to an embodiment of the present invention;

FIG. 17 is a schematic diagram illustrating an “explosive ejection” effect according to an embodiment of the present invention;

FIG. 18 is a schematic diagram illustrating an “explosive propulsion” effect according to an embodiment of the present invention;

FIG. 19 is a schematic diagram illustrating a combination of FIGS. 16-18;

FIG. 20 is a schematic diagram illustrating a “cleanness” of cut obtained by one implementation of the present invention; and

FIG. 21 is a schematic diagram illustrating a roughness of cut obtained by a prior art system.

### DETAILED DESCRIPTION OF THE INVENTION

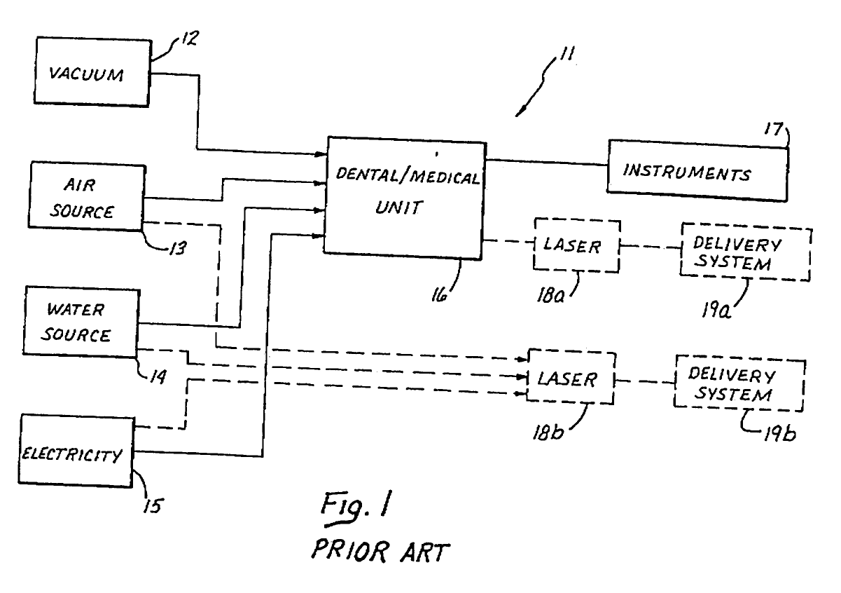

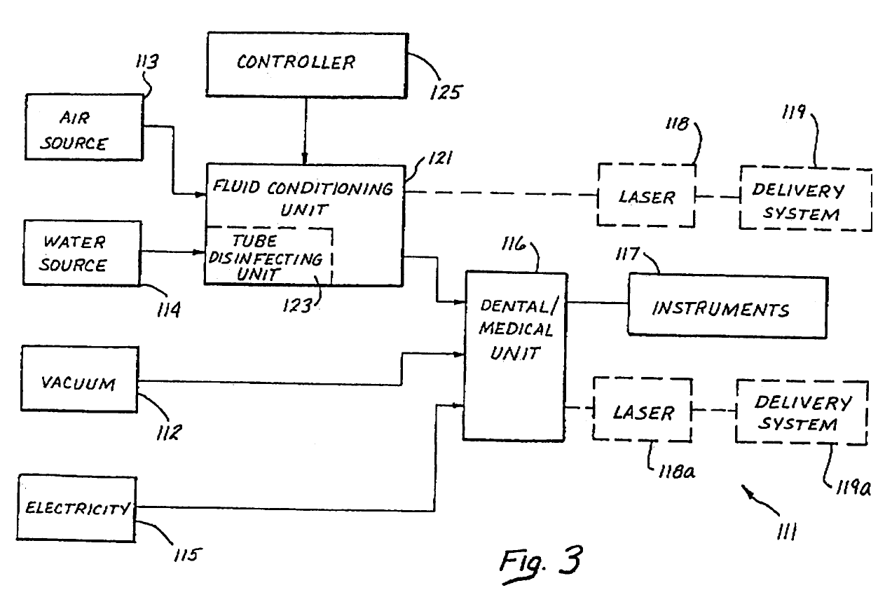

A dental/medical work station 111 of the present invention is shown in FIG. 3, with elements similar to those shown in FIG. 1 proceeded by a “1”. The dental/medical work station 111 comprises a conventional air line 113 and a conventional water line 114 for supplying air and water, respectively. As used herein, the term “water” is intended to encompass various modified embodiments of liquids such as distilled water, deionized water, sterile water, tap water or water that has a controlled number of colony forming units (CFU) for the bacterial count, etc. For instance, drinking water is often chemically treated to a level where there are no more than 500 CFU/ml and in some cases between 100-200 CFU/ml. A vacuum line 112 and an electrical outlet 115 supply negative air pressure and electricity to the dental/medical (e.g., dental or medical) unit 116, similarly to the vacuum 12 and electrical 15 lines shown in FIG. 1. The fluid conditioning unit 121 may, alternatively, be placed between the dental/medical unit 116 and the instruments 117, for example. According to the present invention, the air line 113 and the water line 114 are both connected to a fluid conditioning unit 121.

A controller 125 allows for user inputs, to control whether air from the air line 113, water from the water line 114, or both, are conditioned by the fluid conditioning unit 121. As used herein, mentions of air and/or water are intended to encompass various modified embodiments of the invention, including various biocompatible fluids used with or without the air and/or water, and including equivalents, substitutions, additives, or permutations thereof. For instance, in certain modified embodiments other biocompatable fluids may be used instead of air and/or water. A variety of agents may be applied to the air or water by the fluid conditioning unit 121, according to a configuration of the controller 125, for example, to thereby condition the air or water, before the air or water is output to the dental/medical unit 116. Flavoring agents and related substances, for example, may be used, such as disclosed in 21 C.F.R. Sections 172.510 and 172.515, the details of which are incorporated herein by reference. Colors, for example, may also be used for conditioning, such as disclosed in 21 C.F.R. Section 73.1 to Section 73.3126.

Similarly to the instruments 17 shown in FIG. 1, the instruments 117 may comprise an electrocauterizer, an electromagnetic energy source, a laser, a mechanical drill, a mechanical saw, a canal finder, a syringe, and/or an evacuator. All of these instruments 117 use air from the air line 113 and/or water from the water line 114, which may or may not be conditioned depending on the configuration of the controller 125. Any of the instruments 117 may alternatively be connected directly to the fluid conditioning unit 121 or directly to any of the air 113, water 114, vacuum 112, and/or electric 115 lines. For example, a laser 118 and delivery system 119 is shown in phantom connected to the fluid conditioning unit 121. The laser 118a and delivery system 119a may be connected to the dental/medical unit 116, instead of being grouped with the instruments 117.

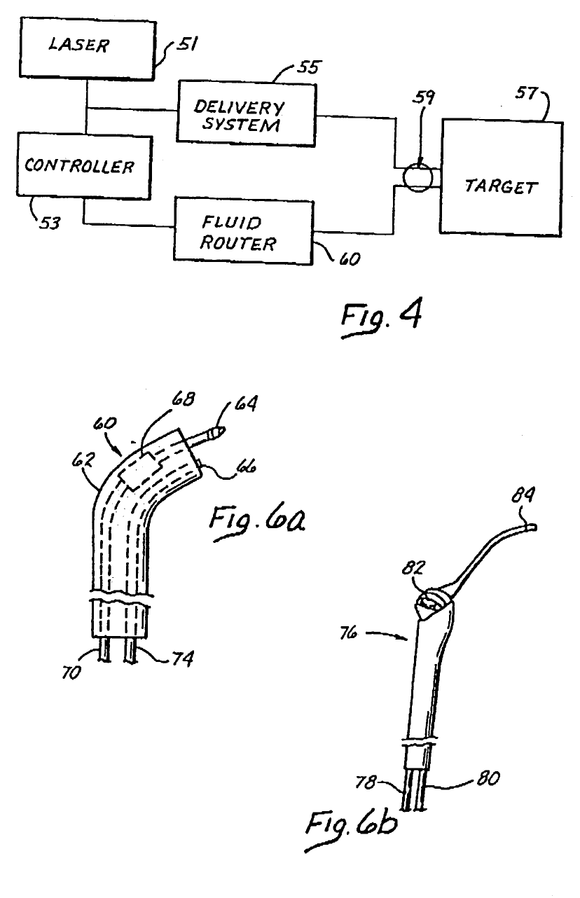

The block diagram shown in FIG. 4 illustrates one embodiment of a laser 51 directly coupled with, for example, the air 113, water 114, and power 115 lines of FIG. 3. A separate fluid conditioning system is used in this embodiment. As an alternative to the laser, or any other tool being connected directly to any or all of the four supply lines 113-115 and having an independent fluid conditioning unit, any of these tools may instead, or additionally, be connected to the dental/medical unit 116 or the fluid conditioning unit 121, or both.

According to the exemplary embodiment shown in FIG. 4, an electromagnetically induced disruptive (e.g., mechanical) cutter is used for cutting. The electromagnetic cutter energy source 51 is connected directly to the outlet 115 (FIG. 3), and is coupled to both a controller 53 and a delivery system 55. The delivery system 55 routes and focuses the laser 51. In the case of a conventional laser system, thermal cutting forces may be imparted onto the target 57. The delivery system 55 can comprise a fiberoptic guide for routing the laser 51 into an interaction zone 59, located above the target surface 57. The fluid router 60 can comprise an atomizer for delivering for example user-specified combinations of atomized fluid particles into the interaction zone 59. The atomized fluid particles are conditioned, according to the present invention, and may comprise flavors, scents, saline, tooth-whitening agents and other actions or agents, as discussed below.

The delivery system 55 for delivering the electromagnetic energy includes a fiberoptic energy guide or equivalent which attaches to the laser system and travels to the desired work site. Fiberoptics or waveguides are typically long, thin and lightweight, and are easily manipulated. Fiberoptics can be made of calcium fluoride (CaF), calcium oxide (CaO2), zirconium oxide (ZrO2), zirconium fluoride (ZrF), sapphire, hollow waveguide, liquid core, TeX glass, quartz silica, germanium sulfide, arsenic sulfide, germanium oxide (GeO2), and other materials. Other delivery systems include devices comprising mirrors, lenses and other optical components where the energy travels through a cavity, is directed by various mirrors, and is focused onto the targeted cutting site with specific lenses.

In the case of a conventional laser, a stream or mist of conditioned fluid is supplied by the fluid router 60. The controller 53 may control various operating parameters of the laser 51, the conditioning of the fluid from the fluid router 60, and the specific characteristics of the fluid from the fluid router 60.

Although the present invention may be used with conventional drills and lasers, for example, an illustrated embodiment includes the above-mentioned electromagnetically induced disruptive cutter. Other embodiments include an electrocauterizer, a syringe, an evacuator, or any air or electrical driver, drilling, filling, or cleaning mechanical instrument.

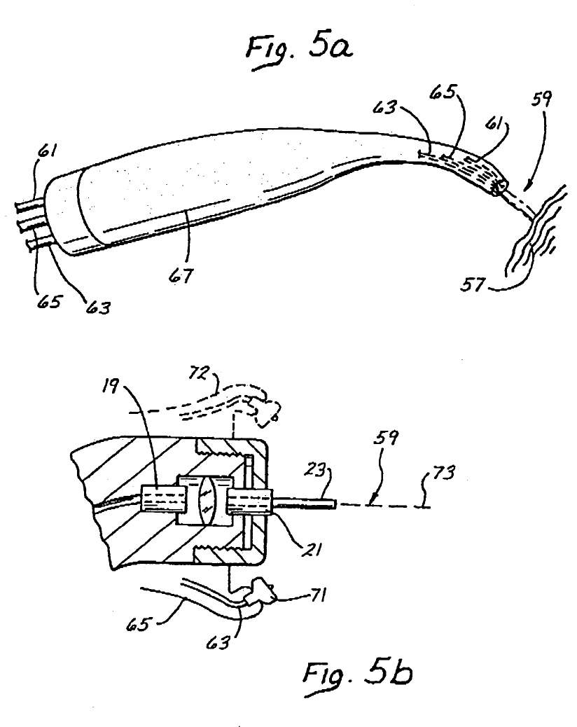

FIG. 10 is a block diagram, similar to FIG. 4 as discussed above, illustrating one electromagnetically induced disruptive cutter of the present invention. The block diagram may be identical to that disclosed in FIG. 4 except the fluid router may not be necessary. As shown in FIG. 10, an electromagnetic energy source 351 is coupled to both a controller 353 and a delivery system 355. The delivery system 355 imparts cutting forces onto the target surface 357. In one implementation, the delivery system 355 comprises a fiberoptic guide 23 (FIG. 5b, infra) for routing the laser 351 through an optional interaction zone 359 and toward the target surface 357.

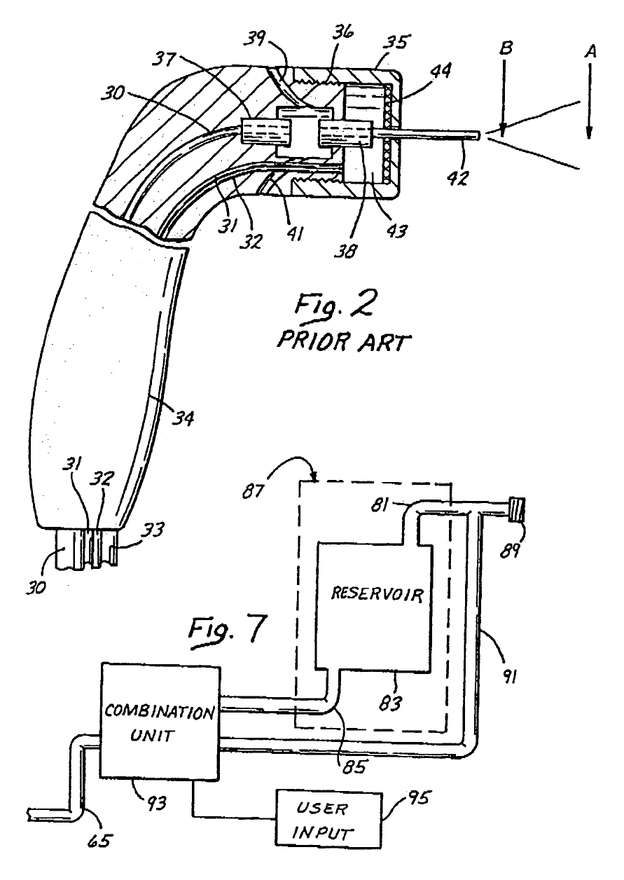

Referring to FIG. 11, an optical cutter according to one aspect of the present invention is shown, comprising, for example, many of the conventional elements of FIG. 2 and further comprising a focusing optic 335 between the two metal cylindrical objects 19 and 21. In modified embodiments, any aspect of the present invention, in addition to being combinable with the embodiment of FIG. 11, may be combined with the structure of FIG. 2 and various modification and equivalents thereof. The focusing optic 335 prevents undesired dissipation of laser energy from the fiber guide tube 5. Although shown coupling two fiber guide tubes having optical axes disposed in a straight line, the focusing optic 335 may be implemented/modified in other embodiments: to couple fiber guide tubes having non parallel optical axes (e.g., two fiber guide tubes having perpendicularly aligned optical axes); to facilitate rotation of one or both of the fiber guide tubes about its respective optical axis; and/or to consist of or comprise one or more of a mirror, pentaprism, or other light directing or transmitting medium. Specifically, energy from the fiber guide tube 5 dissipates slightly before being focused by the focusing optic 335. The focusing optic 335 focuses energy from the fiber guide tube 5 into the fiber guide tube 23. The efficient transfer of laser energy from the fiber guide tube 5 to the fiber guide tube 23 may vitiate any need for the conventional air knife cooling system 33, 39, 41 of FIG. 2, since less laser energy is dissipated. The first fiber guide tube 5 comprises a trunk fiberoptic, which can comprise any of the above-noted fiberoptic materials.

Intense energy emitted from the fiberoptic guide 23 can be generated from a coherent source, such as a laser. In an illustrative embodiment, the laser comprises an erbium, chromium, yttrium, scandium, gallium garnet (Er, Cr:YSGG) solid state laser, which generates light having a wavelength in a range of 2.70 to 2.80 microns. As presently embodied, this laser has a wavelength of approximately 2.78 microns. Fluid emitted from the nozzle 71 (FIG. 5b, infra) comprises water in an illustrated embodiment, other fluids may be used and appropriate wavelengths of the electromagnetic energy source may be selected to allow for high absorption by the fluid. Other possible laser systems include an erbium, yttrium, scandium, gallium garnet (Er:YSGG) solid state laser, which generates electromagnetic energy having a wavelength in a range of 2.70 to 2.80 microns; an erbium, yttrium, aluminum garnet (Er:YAG) solid state laser, which generates electromagnetic energy having a wavelength of 2.94 microns; chromium, thulium, erbium, yttrium, aluminum garnet (CTE:YAG) solid state laser, which generates electromagnetic energy having a wavelength of 2.69 microns; erbium, yttrium orthoaluminate (Er:YALO3) solid state laser, which generates electromagnetic energy having a wavelength in a range of 2.71 to 2.86 microns; holmium, yttrium, aluminum garnet (Ho:YAG) solid state laser, which generates electromagnetic energy having a wavelength of 2.10 microns; quadrupled neodymium, yttrium, aluminum garnet (quadrupled Nd:YAG) solid state laser, which generates electromagnetic energy having a wavelength of 266 nanometers; argon fluoride (ArF) excimer laser, which generates electromagnetic energy having a wavelength of 193 nanometers; xenon chloride (XeCl) excimer laser, which generates electromagnetic energy having a wavelength of 308 nanometers; krypton fluoride (KrF) excimer laser, which generates electromagnetic energy having a wavelength of 248 nanometers; and carbon dioxide (CO2), which generates electromagnetic energy having a wavelength in a range of 9.0 to 10.6 microns.

The delivery system 355 of FIG. 10 can further comprise a fluid output, which may or may not differ from the fluid router 60 of FIG. 4. In exemplary embodiments implementing a fluid output, water can be chosen as a preferred fluid because of its biocompatibility, abundance, and low cost. The actual fluid used may vary as long as it is properly matched (meaning it is highly absorbed) to the selected electromagnetic energy source (i.e. laser) wavelength. In various implementations of the configuration of FIG. 4, the fluid (e.g., fluid particles) can be conditioned. For instance, the fluid can be conditioned to be compatible with the target surface. In one embodiment, the fluid particles comprise water that is conditioned by for example mild chlorination and/or filtering to render the fluid particles compatible (e.g., containing no harmful parasites) with a tooth target surface in a patient's mouth. In other implementations, other types of conditioning may be performed to the fluid as discussed previously. The delivery system 355 can comprise an atomizer for delivering user-specified combinations of atomized fluid particles into the interaction zone 359. The controller 353 controls various operating parameters of the laser 351, and further controls specific characteristics of the user-specified combination of atomized fluid particles output from the delivery system 355, thereby mediating cutting effects on and/or within the target 357.

FIG. 5a shows another embodiment of an electromagnetically induced disruptive cutter, in which a fiberoptic guide 61, an air tube 63, and a fluid tube, such as a water tube, 65 are placed within a hand-held housing 67. Although a variety of connections are possible, the air tube 63 and water tube 65 can be connected to either the fluid conditioning unit 121 or the dental/medical unit 116 of FIG. 3. The fluid tube 65 can be operated under a relatively low pressure, and the air tube 63 can be operated under a relatively high pressure.

According to one aspect of the present invention, either the air from the air tube 63 or the fluid from the fluid tube 65, or both, are selectively conditioned by the fluid conditioning unit 121, as controlled by the controller 125. In one implementation, the laser energy from the fiberoptic guide 61 focuses onto a combination of air and fluid, from the air tube 63 and the fluid tube 65, at the interaction zone 59. Atomized fluid particles in the air and fluid mixture absorb energy from the laser energy of the fiberoptic tube 61, and explode. The explosive forces from these atomized fluid particles can in certain implementations impart disruptive (e.g., mechanical) cutting forces onto the target 57.

Turning back to FIG. 2, a conventional optical cutter focuses laser energy on a target surface at an area A, for example, and in comparison, the electromagnetically induced disruptive cutter of the present invention focuses laser energy into an interaction zone B, for example. The conventional optical cutter uses the laser energy directly to cut tissue, and in comparison, the electromagnetically induced disruptive cutter of the present invention uses the laser energy to expand atomized fluid particles to thus impart disruptive cutting forces onto the target surface. The atomized fluid particles are heated, expanded, and cooled before contacting the target surface. The prior art optical cutter may use a large amount of laser energy to cut the area of interest, and also may use a large amount of water to both cool this area of interest and remove cut tissue.

In contrast, the electromagnetically induced disruptive cutter of the present invention can use a relatively small amount of water and, further, can use only a small amount of laser energy to expand atomized fluid particles generated from the water. According to the electromagnetically induced disruptive cutter of the present invention, additional water may not be needed to cool the area of surgery, since the exploded atomized fluid particles are cooled by exothermic reactions before they contact the target surface. Thus, atomized fluid particles of the present invention are heated, expanded, and cooled before contacting the target surface. The electromagnetically induced disruptive cutter of the present invention is thus capable of cutting without charring or discoloration.

FIG. 5b illustrates another embodiment of the electromagnetically induced mechanical cutter. The atomizer for generating atomized fluid particles comprises a nozzle 71, which may be interchanged with other nozzles (not shown) for obtaining various spatial distributions of the atomized fluid particles, according to the type of cut desired. A second nozzle 72, shown in phantom lines, may also be used. In a simple embodiment, a user controls the air and water pressure entering into the nozzle 71. The nozzle 71 is thus capable of generating many different user-specified combinations of atomized fluid particles and aerosolized sprays. The nozzle 71 is employed to create an engineered combination of small particles of the chosen fluid. The nozzle 71 may comprise several different designs including liquid only, air blast, air assist, swirl, solid cone, etc. When fluid exits the nozzle 71 at a given pressure and rate, it is transformed into particles of user-controllable sizes, velocities, and spatial distributions. The cone angle may be controlled, for example, by changing the physical structure of the nozzle 71. For example, various nozzles 71 may be interchangeably placed on the electromagnetically induced disruptive cutter. Alternatively, the physical structure of a single nozzle 71 may be changed.

The emitted energy may have an output optical energy distribution that may be useful for achieving or maximizing a cutting effect of an electromagnetic energy source, such as a laser, directed toward a target surface. The cutting and/or ablating effects created by the energy may occur on or at the target surface, within the target surface, and/or above the target surface. For instance, using desired optical energy distributions, it is possible to disrupt a target surface by directing electromagnetic energy toward the target surface so that a portion of the energy is absorbed by fluid wherein fluid absorbing the energy may be on the target surface, within the target surface, above the target surface, or a combination thereof.

In certain embodiments, the fluid absorbing the energy may comprise water and/or may comprise hydroxide. When the fluid comprises hydroxide and/or water which highly absorb the electromagnetic energy, molecules within these fluids may begin to vibrate. As the molecules vibrate, the molecules heat and can expand, leading to for example thermal cutting with certain output optical energy distributions. Other thermal cutting or thermal effects may occur by the absorption of the impinging electromagnetic energy by for example other molecules of the target surface. Accordingly, the cutting effects from the energy absorption associated with certain output optical energy distributions may be due to thermal properties (e.g., thermal cutting) and/or by absorptions of the energy by molecules (e.g., water above the target surface) that do not significantly heat the target surface. The use of certain desired optical energy distributions can reduce secondary damage to the target surface, such as charring or burning, in embodiments for example wherein cutting is performed in combination with a fluid output and also in other embodiments that do not use a fluid output. Thus, for example, a portion of the cutting effects caused by the electromagnetic energy may be due to thermal energy, and a portion of the cutting effects may be due to disruptive (e.g., mechanical) forces generated by the molecules absorbing the electromagnetic energy, as discussed herein.

Not only can the cutting effects of the apparatus be mediated by fluid distributions above the target surface, as disclosed above, but the cutting effects may alternatively or additionally be mediated by the absorption of energy by fluid on or within the target surface. In one embodiment of the apparatus, the cutting effects are mediated by effects of energy absorption by a combination of fluid located above the target surface, fluid located on the target surface, or fluid located in the target surface. In one embodiment, about one-third of the impinging electromagnetic energy passes through the fluid particles and impinges onto the target surface, and a portion of that impinging energy can operate to cut or contribute to the cutting of the target surface.

A filter may also be provided with the apparatus to modify electromagnetic energy transmitted from the electromagnetic energy source so that the target surface is disrupted in a spatially different manner at one or more points in time compared to electromagnetic energy that is transmitted to a surface without a filter. The spatial and/or temporal distribution of electromagnetic energy may be changed in accordance with the spatial and/or temporal composition of the filter. The filter may comprise, for example, fluid; and in one embodiment the filter is a distribution of atomized fluid particles the characteristics (e.g., size, distribution, velocity, composition) of which can be changed spatially over time to vary the amount of energy impinging on the target surface. As one example, a filter can be intermittently placed over a target to vary the intensity of the impinging energy to thereby provide a type of pulsed effect. In such an example, a spray or sprays of water can be intermittently applied to intersect the impinging radiation. In some embodiments, utilization of a filter cutting of the target surface may be achieved with reduced, or no, secondary heating/damage that may typically be associated with thermal cutting of prior art lasers that do not have a filter. The fluid of the filter can comprise, for example, water. The outputs from the filter, as well as other fluid outputs, energy sources, and other structures and methods disclosed herein, may comprise any of the fluid outputs and other structures/methods described in U.S. Pat. No. 6,231,567, entitled MATERIAL REMOVER AND METHOD, the entire contents of which are incorporated herein by reference to the extent compatible and not mutually exclusive.

In one embodiment, an output optical energy distribution includes a plurality of high-intensity leading micropulses that impart some high peak amounts of energy that are directed toward a target surface. The energy is directed toward the target surface to obtain the desired cutting effects. For example, the energy may be directed into atomized fluid particles, as discussed above, and the fluid and/or OH molecules present on or in the material of the target surface which in some instances can comprise water, to thereby expand the fluid and induce disruptive cutting forces to or a disruption (e.g., mechanical disruption) of the target surface. The output optical energy distribution may also include one or more trailing micropulses after the maximum leading micropulse that may further help with removal of material. According to the present invention, a single large leading micropulse may be generated or, alternatively, two or more large leading micropulses may be generated. In accordance with one aspect of the present invention, relatively steeper slopes of the pulse and shorter duration of the pulses may lower an amount of residual heat produced in the material.

The output optical energy distribution may be generated by a flashlamp current generating circuit that is configured to generate a relatively narrow pulse, which is on the order of 0.25 to 300 microseconds, for example. Additionally, the full-width half-max value of the optical output energy distribution of the present invention can occur within the first 30 to 70 microseconds, for example, compared to full-width half-max values of the prior art occurring within the first 250 to 300 microseconds. The relatively quick frequency, and the relatively large initial distribution of optical energy in the leading portion of each pulse of the present invention, can result in relatively efficient disruptive cutting (e.g., mechanical cutting). The output optical energy distributions of the present invention can be adapted for cutting, shaping and removing tissues and materials, and further can be adapted for imparting electromagnetic energy into atomized fluid particles over a target surface, or other fluid particles located on or within the target surface. The cutting effect obtained by the output optical energy distributions of the present invention can be both clean and powerful and, additionally, can impart consistent cuts or other disruptive forces onto target surfaces.

By controlling characteristics of the output optical energy, such as pulse intensity, duration, and number of micropulses, the device of the present invention can be adjusted to provide a desired treatment for multiple conditions. In addition, the energy emitted from the devices disclosed herein may be effective to cut a target surface, as discussed above, but may also be effective to remodel a target surface. For example, a surface of a tooth can be remodeled without removing any of the tooth structure. In one embodiment, the output optical energy is selected to have properties that are effective to make a surface of a tooth relatively harder compared to before treatment with the device herein. By making the tooth physically harder, it may become more difficult for bacteria to damage the tooth. Remodeling energy may be particularly effective to inhibit and/or prevent dental carries. In one embodiment, the output optical energy may include a pulse with a relatively longer duration than the pulse described herein that is used for cutting. The pulse may include a series of steep micropulses, as discussed herein, and a longer tail of micropulses where the energy is maintained at a desired level for extended periods of time. In another embodiment, two modes of operation may be utilized, such as, for example, a first pulse as described above with one or more intense micropulses, and a second pulse that has a relatively slower leading and trailing slope. Two mode embodiments may be particularly useful when both cutting and remodeling are desired. Thus, by remodeling a tooth's surface, including the anterior and/or posterior surfaces, the tooth may become harder which may be conducive to preventing tooth decay.

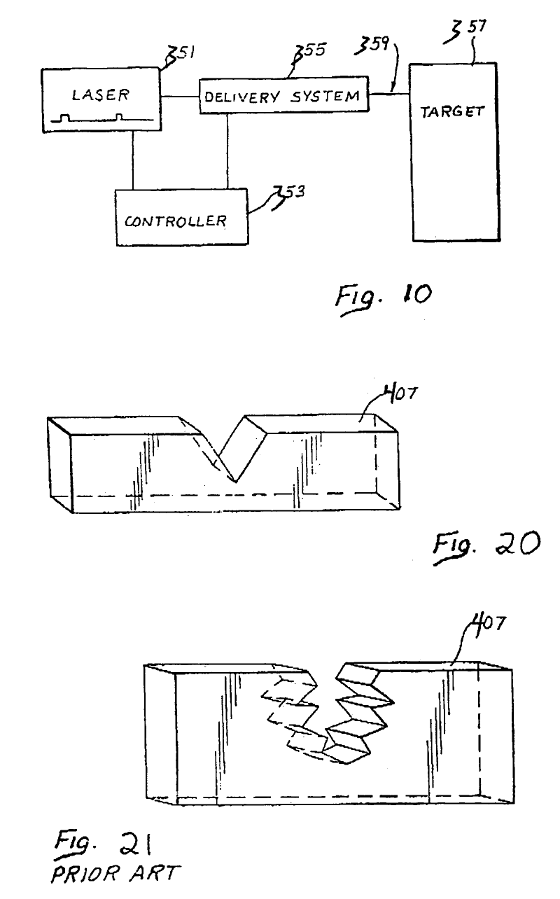

Referring back to the figures, and in particular FIG. 12, a control panel 377 for allowing user-programmability of the atomized fluid particles is illustrated. By changing the pressure and flow rates of the fluid, for example, the user can control the atomized fluid particle characteristics. These characteristics determine absorption efficiency of the laser energy, and the subsequent cutting effectiveness of the electromagnetically induced disruptive cutter. This control panel may comprise, for example, a fluid particle size control 378, a fluid particle velocity control 379, a cone angle control 380, an average power control 381, a repetition rate 382, and a fiber selector 383.

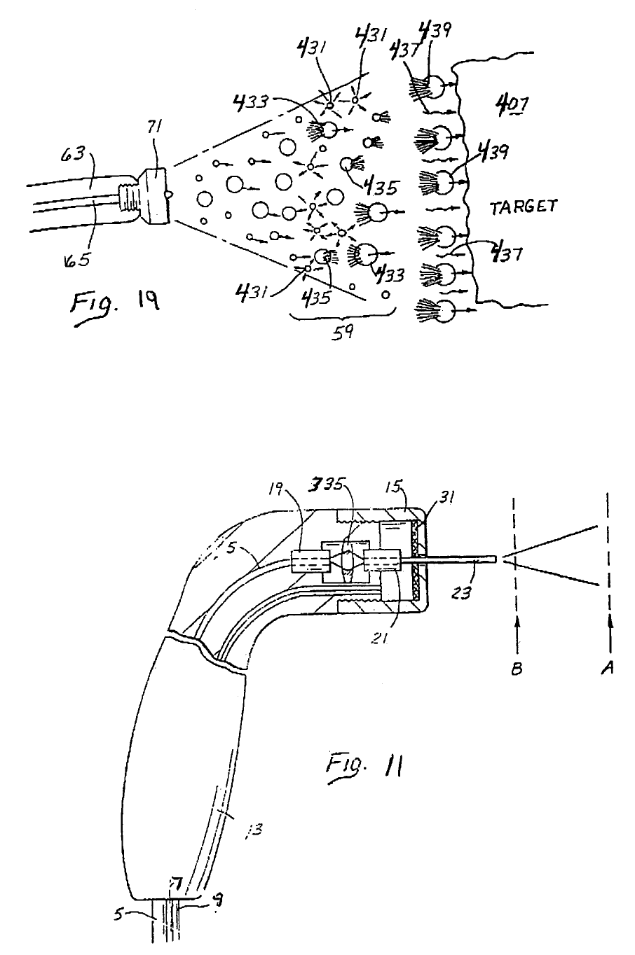

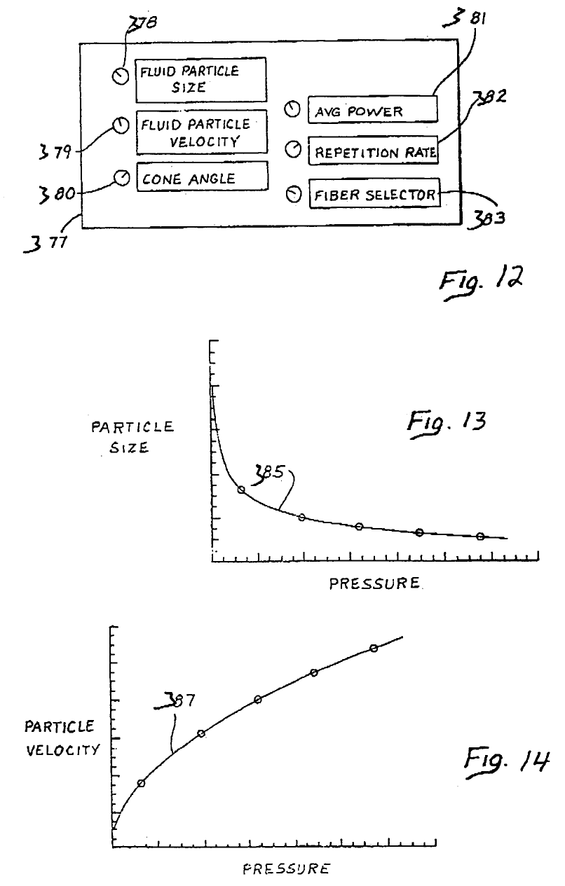

FIG. 13 illustrates a plot 385 of mean fluid particle size versus pressure. According to this figure, when the pressure through the nozzle 71 is increased, the mean fluid particle size of the atomized fluid particles decreases. The plot 387 of FIG. 14 shows that the mean fluid particle velocity of these atomized fluid particles increases with increasing pressure.

According to one implementation of the present invention, materials can be removed from a target surface at least in part by disruptive cutting forces, instead of by conventional (e.g., thermal) cutting forces. In such implementations, energy is used only to induce disruptive forces onto the targeted material. Thus, the atomized fluid particles act as the medium for transforming the electromagnetic energy of the laser into the disruptive (e.g., mechanical) energy required to achieve the disruptive cutting effect of the present invention. The laser energy itself may not be directly absorbed by the targeted material. The disruptive (e.g., mechanical) interaction of the present invention can be safer, faster, and can in certain implementations attenuate or eliminate negative thermal side-effects typically associated with conventional laser cutting systems.

The fiberoptic guide 23 (e.g., FIG. 5b) can be placed into close proximity of the target surface. This fiberoptic guide 23, however, does not actually contact the target surface. Since the atomized fluid particles from the nozzle 71 are placed into the interaction zone 59, the purpose of the fiberoptic guide 23 is for placing laser energy into this interaction zone, as well. A feature of the present invention is the formation of the fiberoptic guide 23 of sapphire. Regardless of the composition of the fiberoptic guide 23, however, another feature of the present invention is the cleaning effect of the air and water, from the nozzle 71, on the fiberoptic guide 23.

Applicants have found that this cleaning effect is optimal when the nozzle 71 is pointed somewhat directly at the target surface. For example, debris from the disruptive cutting can be removed by the spray from the nozzle 71.

Additionally, applicants have found that this orientation of the nozzle 71, pointed toward the target surface, can enhance the cutting efficiency of the present invention. Each atomized fluid particle contains a small amount of initial kinetic energy in the direction of the target surface. When electromagnetic energy from the fiberoptic guide 23 contacts an atomized fluid particle, the spherical exterior surface of the fluid particle acts as a focusing lens to focus the energy into the water particle's interior.

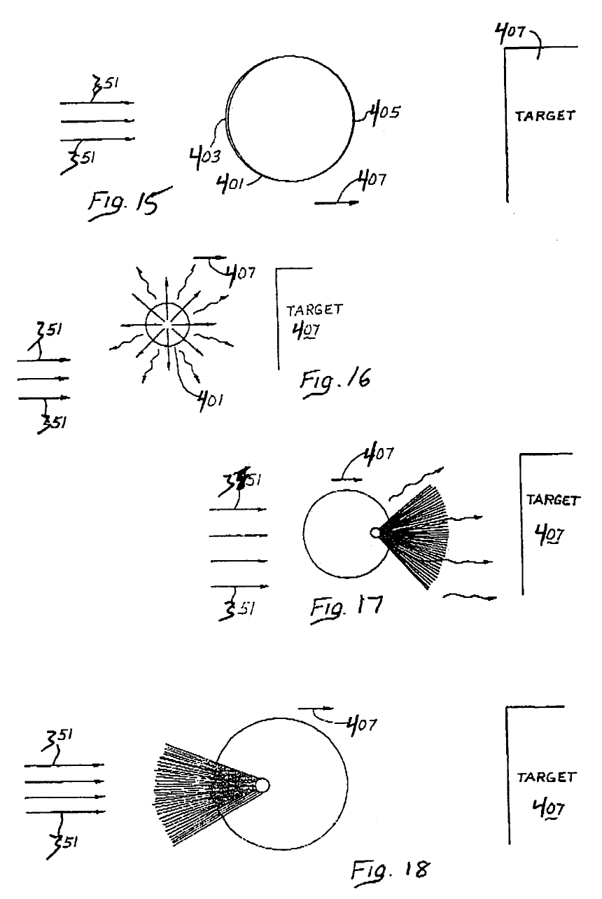

As shown in FIG. 15, the water particle 401 has an illuminated side 403, a shaded side 405, and a particle velocity 407. The focused electromagnetic energy is absorbed by the water particle 401, causing the interior of the water particle to heat and explode rapidly. This exothermic explosion cools the remaining portions of the exploded water particle 401. The surrounding atomized fluid particles further enhance cooling of the portions of the exploded water particle 401. A pressure-wave is generated from this explosion. This pressure-wave, and the portions of the exploded water particle 401 of increased kinetic energy, are directed toward the target surface 407. The incident portions from the original exploded water particle 401, which are now traveling at high velocities with high kinetic energies, and the pressure-wave, impart strong, concentrated, disruptive (e.g., mechanical) forces onto the target surface 407.

These disruptive forces cause the target surface 407 to break apart from the material surface through a “chipping away” action. The target surface 407 does not undergo vaporization, disintegration, or charring. The chipping away process can be repeated by the present invention until the desired amount of material has been removed from the target surface 407. Unlike prior art systems, certain implementations of the present invention may not require a thin layer of fluid. In fact, while not wishing to be limited, a thin layer of fluid covering the target surface may in certain implementations interfere with the above-described interaction process. In other implementations, a thin layer of fluid covering the target surface may not interfere with the above-described interaction process.

FIGS. 16, 17 and 18 illustrate various types of absorptions of the electromagnetic energy by atomized fluid particles. The nozzle 71 can be configured to produce atomized sprays with a range of fluid particle sizes narrowly distributed about a mean value. The user input device for controlling cutting efficiency may comprise a simple pressure and flow rate gauge or may comprise a control panel as shown in FIG. 12, for example. Upon a user input for a high resolution cut, relatively small fluid particles are generated by the nozzle 71. Relatively large fluid particles are generated for a user input specifying a low resolution cut. A user input specifying a deep penetration cut causes the nozzle 71 to generate a relatively low density distribution of fluid particles, and a user input specifying a shallow penetration cut causes the nozzle 71 to generate a relatively high density distribution of fluid particles. If the user input device comprises the simple pressure and flow rate gauge, then a relatively low density distribution of relatively small fluid particles can be generated in response to a user input specifying a high cutting efficiency. Similarly, a relatively high density distribution of relatively large fluid particles can be generated in response to a user input specifying a low cutting efficiency. Other variations are also possible.

These various parameters can be adjusted according to the type of cut and the type of tissue. Hard tissues include tooth enamel, tooth dentin, tooth cementum, bone, and cartilage. Soft tissues, which the electromagnetically induced disruptive cutter of the present invention is also adapted to cut, include skin, mucosa, gingiva, muscle, heart, liver, kidney, brain, eye, and vessels. Other materials may include glass and semiconductor chip surfaces, for example. A user may also adjust the combination of atomized fluid particles exiting the nozzle 71 to efficiently implement cooling and cleaning of the fiberoptic 23 (FIG. 5b), as well. According to an illustrated embodiment, the combination of atomized fluid particles may comprise a distribution, velocity, and mean diameter to effectively cool the fiberoptic guide 23, while simultaneously keeping the fiberoptic guide 23 clean of particular debris which may be introduced thereon by the surgical site.

Looking again at FIG. 15, electromagnetic energy contacts each atomized fluid particle 401 on its illuminated side 403 and penetrates the atomized fluid particle to a certain depth. The focused electromagnetic energy is absorbed by the fluid, inducing explosive vaporization of the atomized fluid particle 401.

The diameters of the atomized fluid particles can be less than, almost equal to, or greater than the wavelength of the incident electromagnetic energy. In each of these three cases, a different interaction occurs between the electromagnetic energy and the atomized fluid particle. FIG. 16 illustrates a case where the atomized fluid particle diameter is less than the wavelength of the electromagnetic energy (d<lambda.). This case causes the complete volume of fluid inside of the fluid particle 401 to absorb the laser energy, inducing explosive vaporization. The fluid particle 401 explodes, ejecting its contents radially. Applicants refer to this phenomena as the “explosive grenade” effect. As a result of this interaction, radial pressure-waves from the explosion are created and projected in the direction of propagation. The direction of propagation is toward the target surface 407, and in one embodiment, both the laser energy and the atomized fluid particles are traveling substantially in the direction of propagation.

The resulting portions from the explosion of the water particle 401, and the pressure-wave, produce the “chipping away” effect of cutting and removing of materials from the target surface 407. Thus, according to the “explosive grenade” effect shown in FIG. 16, the small diameter of the fluid particle 401 allows the laser energy to penetrate and to be absorbed violently within the entire volume of the liquid. Explosion of the fluid particle 401 can be analogized to an exploding grenade, which radially ejects energy and shrapnel. The water content of the fluid particle 401 is evaporated due to the strong absorption within a small volume of liquid, and the pressure-waves created during this process produce the material cutting process.

FIG. 17 shows a case where the fluid particle 401 has a diameter, which is approximately equal to the wavelength of the electromagnetic energy (d approximately equal to lambda). According to this “explosive ejection” effect, the laser energy travels through the fluid particle 401 before becoming absorbed by the fluid therein. Once absorbed, the fluid particle's shaded side heats up, and explosive vaporization occurs. In this case, internal particle fluid is violently ejected through the fluid particle's shaded side, and moves rapidly with the explosive pressure-wave toward the target surface. As shown in FIG. 17, the laser energy is able to penetrate the fluid particle 401 and to be absorbed within a depth close to the size of the particle's diameter. The center of explosive vaporization in the case shown in FIG. 17 is closer to the shaded side 405 of the moving fluid particle 401. According to this “explosive ejection” effect shown in FIG. 17, the vaporized fluid is violently ejected through the particle's shaded side toward the target surface 407.

A third case shown in FIG. 18 is the “explosive propulsion” effect. In this case, the diameter of the fluid particle is larger than the wavelength of the electromagnetic energy (d>lambda). In this case, the laser energy penetrates the fluid particle 401 only a small distance through the illuminated surface 403 and causes this illuminated surface 403 to vaporize. The vaporization of the illuminated surface 403 tends to propel the remaining portion of the fluid particle 401 toward the targeted material surface 407. Thus, a portion of the mass of the initial fluid particle 401 is converted into kinetic energy, to thereby propel the remaining portion of the fluid particle 401 toward the target surface with a high kinetic energy. This high kinetic energy is additive to the initial kinetic energy of the fluid particle 401. The effects shown in FIG. 18 can be visualized as a micro-hydro rocket with a jet tail, which helps propel the particle with high velocity toward the target surface 407. The exploding vapor on the illuminated side 403 thus supplements the particle's initial forward velocity.

The combination of FIGS. 16-18 is shown in FIG. 19. The nozzle 71 produces the combination of atomized fluid particles which are transported into the interaction zone 59. Laser is focused on this interaction zone 59. Relatively small fluid particles 431 explode via the “grenade” effect, and relatively large fluid particles 433 explode via the “explosive propulsion” effect. Medium sized fluid particles, having diameters approximately equal to the wavelength of the laser and shown by the reference number 435, explode via the “explosive ejection” effect. The resulting pressure-waves 437 and exploded fluid particles 439 impinge upon the target surface 407. FIG. 20 illustrates the clean, high resolution cut produced by the electromagnetically induced disruptive cutter of the present invention. Unlike the cut of the prior art shown in FIG. 21, the cut of the present invention can be clean and precise. Among other advantages, this cut can provide an ideal bonding surface, can be accurate, and may not stress remaining materials surrounding the cut.

An illustrated embodiment of light delivery for medical applications of the present invention is through a fiberoptic conductor, because of its light weight, lower cost, and ability to be packaged inside of a handpiece of familiar size and weight to the surgeon, dentist, or clinician. Non-fiberoptic systems may be used in both industrial applications and medical applications, as well. The nozzle 71 is employed to create an engineered combination of small particles of the chosen fluid. The nozzle 71 may comprise several different designs including liquid only, air blast, air assist, swirl, solid cone, etc. When fluid exits the nozzle 71 at a given pressure and rate, it is transformed into particles of user-controllable sizes, velocities, and spatial distributions.

A mechanical drill 60 is shown in FIG. 6a, comprising a handle 62, a drill bit 64, and a water output 66. The mechanical drill 60 comprises a motor 68, which may be electrically driven, or driven by pressurized air.

When the motor 68 is driven by air, for example, the fluid enters the mechanical drill 60 through the first supply line 70. Fluid entering through the first supply line 70 passes through the motor 68, which may comprise a turbine, for example, to thereby provide rotational forces to the drill bit 64. A portion of the fluid, which may not appeal to a patient's taste and/or smell, may exit around the drill bit 64, coming into contact with the patient's mouth and/or nose. The majority of the fluid exits back through the first supply line 70.

In the case of an electric motor, for example, the first supply line 70 provides electric power. The second supply line 74 supplies fluid to the fluid output 66. The water and/or air supplied to the mechanical drill 60 may be selectively conditioned by the fluid conditioning unit 121, according to the configuration of the controller 125.

The syringe 76 shown in FIG. 6b comprises an air input line 78 and a water input line 80. A user control 82 is movable between a first position and a second position. The first position supplies air from the air line 78 to the output tip 84, and the second position supplies water from the water line 80 to the output tip 84. Either the air from the air line 78, the water from the water line 80, or both, may be selectively conditioned by the fluid conditioning unit 121, according to the configuration of the controller 125, for example.

Turning to FIG. 7, a portion of the fluid conditioning unit 121 (FIG. 3) is shown. This fluid conditioning unit 121 can be adaptable to existing water lines 114, for providing conditioned fluid to the dental/medical unit 116 as a substitute for regular tap water in drilling and cutting operations, for example. The interface 89 connects to an existing water line 114 and feeds water through the fluid-in line 81 and the bypass line 91. The reservoir 83 accepts water from the fluid-in line 81 and outputs conditioned fluid to the fluid-out line 85. The fluid-in line 81, the reservoir 83, and the fluid-out line 85 together comprise a fluid conditioning subunit 87.

Conditioned fluid is output from the fluid conditioning subunit 87 into the combination unit 93. The fluid may be conditioned by conventional means, such as the addition of a tablet, liquid syrup, or a flavor cartridge. Also input into the combination unit 93 is regular water from the bypass line 91. A user input 95 into the controller 125, for example, determines whether fluid output from the combination unit 93 into the fluid tube 65 comprises only conditioned fluid from the fluid-out line 85, only regular water from the bypass line 91, or a combination thereof. The user input 95 comprises a rotatable knob, a pedal, or a foot switch, operable by a user, for determining the proportions of conditioned fluid and regular water. These proportions may be determined according to the pedal or knob position. In the pedal embodiment, for example, a full-down pedal position corresponds to only conditioned fluid from the fluid outline 85 being output into the fluid tube 65, and a full pedal up position corresponds to only water from the bypass line 91 being output into the fluid tube 65. The bypass line 91, the combination unit 93, and the user input 95 provide versatility, but may be omitted, according to preference. A simple embodiment for conditioning fluid would comprises only the fluid conditioning subunit 87.

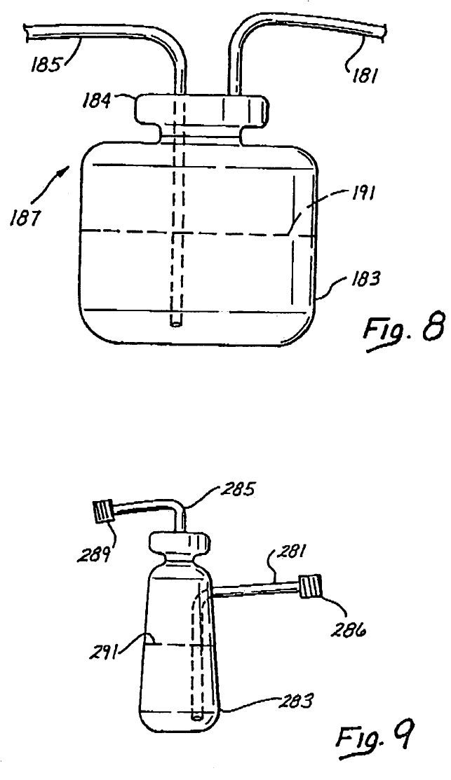

An alternative embodiment of the fluid conditioning subunit 87 is shown in FIG. 8. The fluid conditioning subunit 187 inputs air from air line 113 via an air input line 181, and outputs conditioned fluid via a fluid output line 185. The fluid output line 185 can extend vertically down into the reservoir 183 into the fluid 191 located therein. The lid 184 may be removed and conditioned fluid inserted into the reservoir 183. Alternatively, a solid or liquid form of fluid conditioner may be added to water already in the reservoir 183. The fluid can be conditioned, using either a scent fluid drop or a scent tablet (not shown), and may be supplied with fungible cartridges, for example.

The fluid 191 within the reservoir 183 may be conditioned to achieve a desired flavor, such as a fruit flavor or a mint flavor, or may be conditioned to achieve a desired scent, such as an air freshening smell. In one embodiment wherein the reservoir is conditioned to achieve a desired flavor, the flavoring agent for achieving the desired flavor does not consist solely of a combination of saline and water and does not consist solely of a combination of detergent and water. A conditioned fluid having a scent, a scented mist, or a scented source of air, may be particularly advantageous for implementation in connection with an air conditioning unit, as shown in FIG. 9 and discussed below. In addition to flavor and scents, other conditioning agents may be selectively added to a conventional water line, mist line, or air line. For example, an ionized solution, such as saline water, or a pigmented solution may be added, as discussed below. Additionally, agents may be added to change the density, specific gravity, pH, temperature, or viscosity of water and/or air supplied to a drilling or cutting operation. These agents may include a tooth-whitening agent for whitening a tooth of a patient. The tooth-whitening agent may comprise, for example, a peroxide, such as hydrogen peroxide, urea peroxide, or carbamide peroxide. The tooth-whitening agent may have a viscosity on an order of about 1 to 15 centipoises (cps). Medications, such as antibiotics, steroids, anesthetics, anti-inflammatories, disinfectants, adrenaline, epinephrine, or astringents may be added to the water and/or air used in a drilling or cutting operation. In one embodiment the medication does not consist solely of a combination of saline and water and does not consist solely of a combination of detergent and water. For example, an astringent may be applied to a surgical area, via the water line to reduce bleeding. Vitamins, herbs, or minerals may also be used for conditioning the air or water used in a cutting or drilling procedure. An anesthetic or anti-inflammatory applied to a surgical wound may reduce discomfort to the patient or trauma to the wound, and an antibiotic or disinfectant may prevent infection to the wound.

The air conditioning subunit shown in FIG. 9 is connectible into an existing air line 113, via interfaces 286 and 289. Conventional air enters the conditioning subunit via the air input line 281, and exits an air output line 285. The air input line 281 can extend vertically into the reservoir 283 into a fluid 291 within the reservoir 283. The fluid 291 can be conditioned, using either a scent fluid drop or a scent tablet (not shown). The fluid 291 may be conditioned with other agents, as discussed above in the context of conditioning water. According to the present invention, water in the water line 31 or air in the air line 32 of a conventional laser cutting system (FIG. 2) is conditioned. Either the fluid tube 65 or the air tube 63 (FIG. 5a) of the electromagnetically induced disruptive cutter is conditioned. In addition to laser operations, the air and/or water of a dental drilling, irrigating, suction, or electrocautery system may also be conditioned.

Many of the above-discussed conditioning agents may change the absorption of the electromagnetic energy into the atomized fluid particles in the electromagnetically induced disruptive (e.g., mechanical) cutting environment of the illustrated embodiment. Accordingly, the type of conditioning may effect the cutting power of an electromagnetic or an electromagnetically induced disruptive cutter. Thus, in addition to the direct benefits achievable through these various conditioning agents discussed above, such as flavor or medication, these various conditioning agents further provide versatility and programmability to the type of cut resulting from the electromagnetic or electromagnetically induced disruptive cutter. For example, introduction of a saline solution will reduce the speed of cutting. Such a biocompatible saline solution may be used for delicate cutting operations or, alternatively, may be used with a higher laser-power setting to approximate the cutting power achievable with regular water.

Pigmented fluids may also be used with the electromagnetic or the electromagnetically induced disruptive cutter, according to the present invention. The electromagnetic energy source may be set for maximum absorption of atomized fluid particles having a certain pigmentation, for example. These pigmented atomized fluid particles may then be used to achieve the disruptive cutting. A second water or mist source may be used in the cutting operation, but since this second water or mist is not pigmented, the interaction with the electromagnetic energy source is minimized. As just one example of many, this secondary mist or water source could be flavored.

According to another configuration, the atomized fluid particles may be unpigmented, and the electromagnetic or the electromagnetically induced energy source may be set to provide maximum energy absorption for these unpigmented atomized fluid particles. A secondary pigmented fluid or mist may then be introduced into the surgical area, and this secondary mist or water would not interact significantly with the electromagnetic energy source. As another example, a single source of atomized fluid particles may be switchable between pigmentation and non-pigmentation, and the electromagnetic energy source may be set to be absorbed by one of the two pigment states to thereby provide a dimension of controllability as to exactly when cutting is achieved.

In another embodiment, the source of atomized fluid particles may comprise a tooth whitening agent that is adapted to whiten a tooth of a patient. The tooth-whitening agent may comprise, for example, a peroxide, such as hydrogen peroxide, urea peroxide, or carbamide peroxide. The tooth-whitening agent may have a viscosity on an order of about 1 to 15 cps. The source of atomized fluid particles is switchable by a switching device between a first configuration wherein the atomized fluid particles comprise the tooth-whitening agent and a second configuration wherein the atomized fluid particles do not comprise the tooth-whitening agent. In this configuration, the electromagnetic or electromagnetically induced energy source may comprise, for example, a laser that is operable between an on condition and an off condition, independently of the configuration of the switching device. Thus, regardless of whether the switching device is in the first configuration or the second configuration, the laser can be operated in either the on or off condition.

Disinfectant may be added to an air or water source in order to combat bacteria growth within the air and water lines, and on surfaces within a dental operating room. As used herein, the term “disinfectant” is intended to encompass various modified embodiments of the present invention, including those using disinfectants having one or more of chlorine dioxide, peroxide, hydrogen peroxide, alkaline peroxides, iodine, peracetic acid, acetic acid, chlorite, sodium hypochlorite, citric acid, chlorohexadine gluconate, silver ions, copper ions, equivalents thereof, and combinations thereof. The air and water lines of the dental/medical unit 116, for example, may be periodically flushed with a disinfectant selected by the controller 125 and supplied by the fluid conditioning unit 121. An accessory tube disinfecting unit 123 may accommodate disinfecting cartridges and perform standardized or preprogrammed periodic flushing operations.

Even in a dental or medical procedure, an appropriate disinfectant may be used. The disinfectant may be applied at the end of a dental procedure as a mouthwash, for example, or may be applied during a medical or dental procedure. The air and water used to cool the tissue being cut or drilled within the patient's mouth, for example, is often vaporized into the air to some degree. According to the present invention, a conditioned disinfectant solution will also be vaporized with air or water, and condensate onto surfaces of the dental equipment within the dental operating room. Any bacteria growth on these moist surfaces is significantly attenuated, as a result of the disinfectant on the surfaces.

While this invention has been described with respect to various specific examples and embodiments, it is to be understood that the invention is not limited thereto and that it can be variously practiced with the scope of the following claims. Multiple variations and modification to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. Accordingly, the present invention is not intended to be limited by the disclosed embodiments, but is to be defined by reference to the appended claims.

1. An apparatus using conditioned fluid to treat a target, comprising:
a fluid output pointed in a general direction of an interaction zone, the fluid output being constructed to place fluid particles into the interaction zone, the interaction zone being defined as a volume above the target and the fluid particles being conditioned to be compatible with the target; and
an electromagnetic energy source pointed in a direction of the interaction zone, the electromagnetic energy source being constructed to deliver into the interaction zone a peak concentration of electromagnentic energy that is greater than a concentration of electromagnetic energy delivered onto the target, the electromagnetic energy having a wavelength which is substantially absorbed by the fluid particles in the interaction zone, the absorption of the electromagnetic energy by the fluid particles causing the fluid particles to expand and impart disruptive forces onto the target.

2. The apparatus of claim 1, wherein:
the apparatus is constructed to place fluid on the target; and
electromagnetic energy delivered by the electromagnetic energy source is at least partially absorbed by fluid on the target.

3. The apparatus of claim 2, wherein the electromagnetic energy delivered by the electromagnetic energy source is at least partially absorbed by fluid located within the target.

4. The apparatus of claim 1, wherein electromagnetic energy delivered by the electromagnetic energy source is at least partially absorbed by fluid within the target.

5. The apparatus of claim 1, wherein:
the fluid output is constructed to place the fluid particles into the interaction zone as atomized fluid particles; and
electromagnetic energy is substantially absorbed by the atomized fluid particles in the interaction zone to impart the disruptive forces onto the target.

6. The apparatus of claim 3, wherein at least some of the fluid within the target that absorbs the electromagnetic energy is not supplied from the apparatus.

7. The apparatus of claim 6, wherein:
the target comprises hard or soft tissue; and
the fluid within the target comprises water.

8. The apparatus of claim 1, wherein the electromagnetic energy source comprises one of an Er:YAG, an Er:YSGG, an Er, Cr:YSGG and a CTE:YAG.

9. The apparatus of claim 1, wherein the target surface comprises one of tooth, bone, cartilage and skin.

10. The apparatus of claim 1, wherein the electromagnetic energy source comprises one of a wavelength within a range from about 2.69 to about 2.80 microns and a wavelength of about 2.94 microns.

11. The apparatus of claim 1, comprising a filter, which comprises fluid that is output from the fluid output, wherein the filter absorbs a portion of the energy generated by the electromagnetic energy source.

12. The apparatus of claim 11, wherein the fluid comprises atomized particles of water.

13. The apparatus of claim 1, wherein the disruption of the target is caused in part by energy generated by the electromagnetic energy source other than the energy absorbed by the fluid.

14. The apparatus of claim 1, wherein the electromagnetic energy source comprises an erbium, yttrium, scandium gallium garnet (Er:YSGG) solid state laser or an erbium, yttrium, aluminum garnet (Er:YAG) solid state laser.
